(19) United States
(12) Patent Application Publication
(10) Pub. No.: US 2022/0151444 A1
(43) Pub. Date: May 19, 2022

(54) SYSTEMS AND METHODS TO PROVIDE TOWELETTE UNITS

(71) Applicant: Sweetums, Inc., Carlsbad, CA (US)

(72) Inventor: Alisa Brady, Carlsbad, CA (US)

(21) Appl. No.: 17/129,046

(22) Filed: Dec. 21, 2020

Related U.S. Application Data
(60) Provisional application No. 63/115,734, filed on Nov. 19, 2020.

(51) Int. Cl. A47K 10/32

(52) U.S. Cl. CPC A47K 10/32; A47K 2010/3266

(57) ABSTRACT

A towelette unit and method to provide the towelette unit are disclosed herein. The towelette unit comprising: at least a lubricating agent, a flavor carrier, and a disposable cloth. The lubricating agent and flavor carrier may be combined to create a mixture. the lubricating agent may reduce friction between body tissues. The flavor carrier may facilitates transmitting a flavoring agent to a body surface upon contact. The disposable cloth may retain the mixture for a subsequent single-use event. The single-use event may include wiping a body surface with the disposable cloth to transfer the mixture to the body surface. The flavoring agent imparts a pleasant taste to the surface upon which the wipe is used.

### TECHNICAL FIELD

Lubricating towelettes and mixtures. Flavoring and perfume towelettes and mixtures.

### BACKGROUND

Individuals may desire to easily cleanse, flavor, and lubricate a body surface prior to particular activities.

### SUMMARY

One aspect of the present disclosure is related to a towelette unit that provides lubrication and flavor to a body surface upon contact with the body surface. The towelette unit may comprise a flavor carrier, a lubricating agent, a disposable cloth, and/or other components. Flavor carrier may facilitate transmitting a flavoring agent to the body surface upon contact. Lubricating agent may reduce friction between body tissues such as body surfaces. At least the flavoring carrier and the lubricating agent may be combined to create a mixture. The disposable cloth may be configured to retain the mixture for a subsequent single-use event. The single-use event may include wiping the body surface with the disposable cloth.

These and other features, and characteristics of the present technology, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention. As used in the specification and in the claims, the singular form of ‘a’, ‘an’, and ‘the’ include plural referents unless the context clearly dictates otherwise.

### BRIEF DESCRIPTIONS OF THE DRAWINGS

FIG. 1-5 illustrates a towelette unit, in accordance with one or more implementations.

FIG. 6 illustrates a method to provide the towelette unit, in accordance with one or more implementations.

FIG. 7 illustrates a towelette container configured to stored towelette units individually, in accordance with one or more implementations.

### DETAILED DESCRIPTION

The following description recites various aspects and embodiments of the inventions disclosed herein. No particular invention is intended to define the scope of the invention. Rather, the embodiments provide non-limiting examples of various compositions, and methods that are included within the scope of the claimed inventions. The description is to be read from the perspective of one of ordinary skill in the art. Therefore, information that is well known to the ordinarily skilled artisan is not necessarily included.

The following terms and phrases have the meanings indicated below, unless otherwise provided herein. This disclosure may employ other terms and phrases not expressly defined herein. Such other terms and phrases shall have the meanings they would possess within the context of this disclosure to those of ordinary skill in the art. In some instances, a term or phrase may be defined in the singular or plural. In such instances, it is understood that any term in the singular may include its plural counterpart and vice versa, unless expressly indicated to the contrary.

As used herein, the singular forms “a,” “an,” and “the” include plural referents unless the context clearly indicates otherwise. For example, reference to “a substituent” encompasses a single substituent as well as two or more substituents, and the like.

As used herein, “for example,” “for instance.” “such as,” or “including” are meant to introduce examples that further clarify more general subject matter. Unless otherwise expressly indicated, such examples are provided only to aid in understanding embodiments illustrated in the present disclosure and are not meant to be limiting in any fashion. Nor do these phrases indicate any kind of preference for the disclosed embodiment.

Male and female genitalia can be a source of pleasure for individuals and their partners. There are many methods for stimulation and sexual arousal. Among these methods are licking or sucking the genitalia. While this is pleasurable and sexually arousing to the recipient it may be difficult for the one engaging in the licking and sucking because of odors and/or tastes. Refreshing the genitals and the surrounding area may alleviate the odors and/or tastes and can remove any surface germs, thus providing a more pleasurable environment for the partner whose mouth is on and near the genitals.

Personal lubricants generally work by supplying water to a body surface in a gelled or viscous form. By way of non-limiting example, personal lubricants may include a water-soluble polymer such as a water-soluble cellulose derivative or other water-soluble polymers, such as polyvinylpyrrolidone, polyvinyl alcohol and/or other ingredients. With application of the personal lubricants to the body surface, the personal lubricants retain a supply of water on the body surface so that the water retained provides lubrication or slippage.

Typically, personal lubricants are stored in and dispensed from tubes. The tubes may be collapsible upon squeezing and allow the personal lubricants to be squeezed from the tubes. There are often issues with the tubes. The tubes may be messy, difficult to apply proper pressure to dispense an amount of personal lubricant desired, difficult to store properly for preservation, and/or other issues.

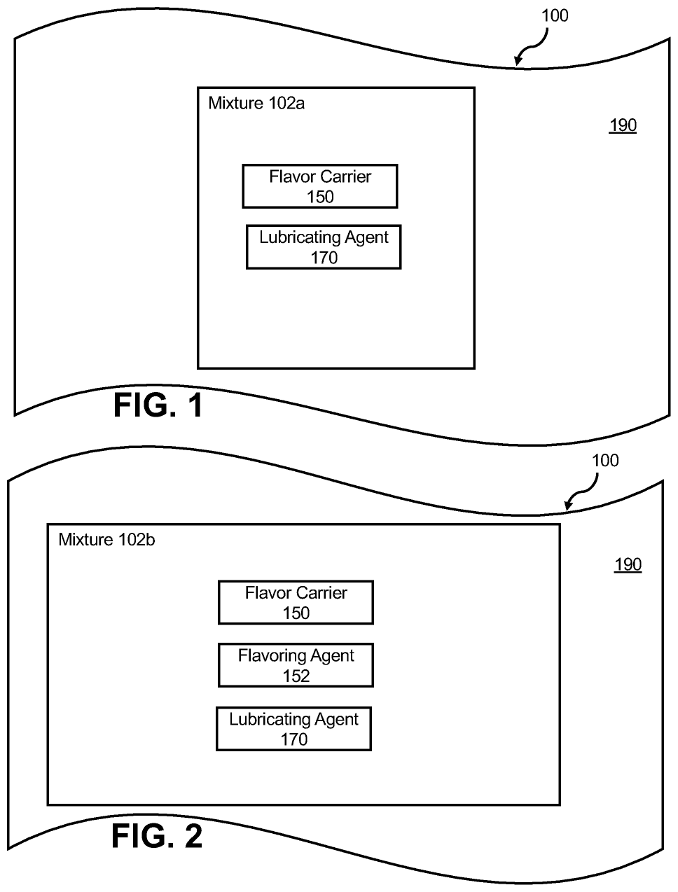

FIG. 1 illustrates a towelette unit 100, in accordance with one or more implementations. The present disclosure forgoes the use of tubes and instead the personal lubricant may be carried in the towelette unit 100. Towelette unit 100 may comprise of one or more of a flavor carrier 150, a lubricating agent 170, a disposable cloth 190, and/or other components. At least the flavoring carrier 150, the lubricating agent 170, and/or other components may be combined to create a mixture 102a.

Flavor carrier 150 may facilitate transmitting a flavoring agent 152 (e.g., flavoring agent 152 of FIG. 2) to a body surface upon contact. As used herein, “flavor carrier 150” may refer to a material which is substantially neutral from flavoring agent 152 insofar as it does not significantly alter the organoleptic properties of the flavoring agent 152 or ingredients thereof. Flavor carrier 150 may be a liquid or a solid. Flavor carrier 150 that is liquid may include, for instance, an emulsifying system, i.e., a solvent and a surfactant system, or a solvent commonly used in flavors. A detailed description of the nature and type of solvents commonly used in flavor cannot be exhaustive. Solvents used in flavor may include, for instance, propylene glycol, triacetine, caprylic/capric triglyceride (neobee), triethyl citrate, benzylic alcohol, ethanol, vegetable oils such as Linseed oil, sunflower oil or coconut oil, glycerol, and/or other solvents for flavor.

Lubricating agent 170, also referred to as personal lubricant, may reduce friction between body tissues. The body tissues may include body surfaces of users of the towelette unit 100. Lubricating agent 170 may refer to specialized lubricants that serve to reduce friction between body tissues. In particular, personal lubricants may be used to provide lubrication or slippage during intercourse activities. For example, personal lubricants may be used to increase pleasure or reduce pain during the intercourse activities, and may help in reducing dryness of body surfaces involved in the intercourse activities. In some implementations, lubricating agent 170 may include one or more humectants to aid water retention, and thus increase the lubrication provided by the lubricating agent 170 and/or increase duration of the lubrication.

Disposable cloth 190 may be configured to retain the mixture 102a for a subsequent single-use event. The disposable cloth 190 may be comprised of nonwoven fabrics, cotton, wood pulp, rayon fibers, and/or other fabrics. In some implementations, the non-woven fabrics may comprise of polyester, polypropylene, and/or other elements. In some implementations, the disposable cloth 190 may be configured of fiber-based textile with fibers that form an outward raised/embossed texture on a first side and/or an opposite side of the first side. In some implementations, the disposable cloth 190 may be made from biodegradable material so that the disposable cloth 190 will degrade after being disposed of.

In some implementations, the mixture 102a may be a solution that is retained by the disposable cloth 190. In some implementations, the mixture 102a may be a dry mixture that is coupled to the disposable cloth. The dry mixture coupled with the disposable cloth 190 may be less prone to degradation or loss of effectiveness. Upon application of a liquid (e.g., water) to the dry mixture coupled with the disposable cloth 190, the solution may be created and retained by the disposable cloth 190 for the single-use event. In some implementations, the dry mixture and the disposable cloth 190 may be compressed and may expand upon the application of the liquid.

The single-use event may include wiping the body surface with the disposable cloth 190 so that the solution of at least the lubricating agent 170, flavor carrier 150, and/or other components transfers to the body surface that contacted the disposable cloth 190. Thus, the disposable cloth 190 may be utilized as an applicator for the solution to the body surface.

FIG. 2 illustrates a towelette unit, in accordance with one or more implementations. Towelette unit 100 (the same as or similar to FIG. 1) may comprise of disposable cloth 190 (the same as or similar to that of FIG. 1) that retains a mixture 102b. Mixture 102b may comprise of a combination of flavor carrier 150 and lubricating agent 170 and the various implementations described thereof in FIG. 1 that may be combined, similar to mixture 102a of FIG. 1. Mixture 102b may further comprise flavoring agent 152 that may include one or more flavoring co-ingredients and/or other ingredients to provide one or more tastes. The one or more flavoring co-ingredients may be one or more compounds used in flavoring mixtures/compositions to impart a hedonic effect. In other words, the flavoring co-ingredients may be able to impart or modify a taste (and/or an odor) of the mixture so that a taste and/or an odor of the mixture is positive and/or pleasant, and not merely a taste or an odor as must be recognized by a person skilled in the art. In some implementations, the positive and/or pleasant taste may include a sweet taste (e.g., a particular fruit), a sour taste, a salty taste, a bitter taste, a savory taste, and/or other tastes. In some implementations, the positive and/or pleasant odor may include floral, sweet (e.g., fruity, chocolate, vanilla), minty, citrus, and/or other odors. In some implementations, flavoring agent 152 may be utilized to moisturize a body surface upon contact and transfer to the body surface.

In some implementations, flavoring agent 152 may include one or more sweeteners. As used herein “sweeteners” may refer to compounds both natural and artificial that impart a sweet taste and/or a sweet odor. By way of non-limiting example, the sweeteners may include common saccharide sweeteners, e.g., sucrose, fructose (e.g., D-fructose), glucose (e.g., D-glucose); sweetener compositions comprising natural sugars, such as stevia (all types and grades), corn syrup (including high fructose corn syrup) or other syrups or sweetener concentrates derived from natural fruit and vegetable sources; semisynthetic “sugar alcohol” sweeteners such as erythritol, isomalt, lactitol, mannitol, sorbitol, xylitol, maltodextrin, glycerol, threitol, arabitol, ribitol, and dulcitol; artificial sweeteners such as miraculin, aspartame, superaspartame, saccharin, saccharin-sodium salt, acesulfame-K, cyclamate, sodium cyclamate, and alitame; other sweeteners such as trehalose, melizitose, melibiose, raffinose, palatinose, lactulose, cyclamic acid, mogroside, tagatose (e.g., D-tagatose), maltose, galactose (e.g., D-galactose), L-rhamnose, D-sorbose, maunose (e.g., D-mannose), lactose, L-arabinose, D-ribose, D-glyceraldehyde, curculin, brazzein, mogroside, Neohesperidin dihydrochalcone (NHDC), neotame and other aspartame derivatives, D-tryptophan, D-leucine, D-threonine, glycine, D-asparagine, D-phenylalanine, L-proline, maltitol, hydrogenated glucose syrup (HGS), magap, sucralose, lugduname, sucrononate, sucrooctate, monatin, phyllodulcin, hydrogenated starch hydrolyzate (HSH), stevioside, rebaudioside A, rebaudioside D, rebadioside M, and other sweet Stevia based glycosides, lo han guo, thaumatin, monellin, carrelameand and other guanidine-based sweeteners, and/or other sweeteners. As such, flavoring agent 152 may be provided by flavor carrier 150.

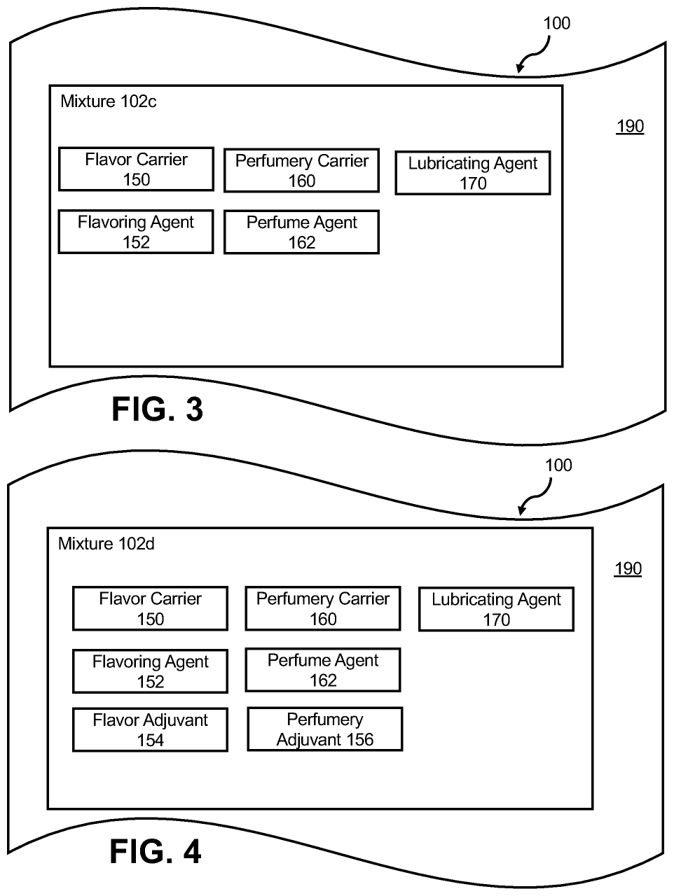

FIG. 3 illustrates a towelette unit, in accordance with one or more implementations. Towelette unit 100 (the same as or similar to FIG. 1-2) may comprise of disposable cloth 190 (the same as or similar to that of FIG. 1-2) that retains a mixture 102c. Mixture 102c may comprise of a combination of flavor carrier 150 and lubricating agent 170 and the various implementations described thereof in FIG. 1, similar to mixture 102a of FIG. 1, and flavoring agent 152 and the various implementations described thereof in FIG. 2. Mixture 102c may further comprise a perfumery carrier 160 and a perfume agent 162, and/or other components.

Perfumery carrier 160 may be the same as or similar to flavor carrier 150 as previously described. Solvents used in perfumery may include, for example, butylene or propylene glycol, glycerol, dipropyleneglycol and its monoether, 1,2,3-propanetriyl triacetate, dimethyl glutarate, dimethyl adipate 1,3-diacetyloxypropan-2-yl acetate, diethyl phthalate, isopropyl myristate, benzyl benzoate, benzyl alcohol, 2-(2-ethoxyethoxy)-l-ethanol, tri-ethyl citrate, ethanol, water/ethanol mixtures, limonene or other terpenes, isoparaffins such as those known under the trademark Isopar (origin: Exxon Chemical) or glycol ethers and glycol ether esters such as those known under the trademark Dowanol (origin: Dow Chemical Company), or hydrogenated castors oils such as those known under the trademark Cremophor RH 40 (origin: BASF) or mixtures thereof, and/or other solvents for perfumery.

Perfume agent 162 may be similar to flavoring agent 152. Perfume agent 162 may include one or more perfuming co-ingredients and/or other ingredients to provide one or more odors. The one or more perfuming co-ingredients may be one or more compounds used in perfuming mixtures/compositions to impart a hedonic effect. In other words, the perfuming co-ingredients may be able to impart or modify an odor of the mixture so that an odor of the mixture is positive and/or pleasant (e.g., sweet odor, fruity odor, minty odor, etc.) similar to the positive and/or pleasant tastes, and not merely an odor as must be recognized by a person skilled in the art.

Perfume agent 162 may be provided by perfumery carrier 160. In some implementations, the flavoring agent 152 and the perfume agent 162 may be the same co-ingredient. In some implementations, individual ones of the flavoring co-ingredients or perfuming co-ingredients may impart a single taste and no odor, or a single odor and no taste.

The nature and type of the flavoring co-ingredients included in the mixture 102b of FIG. 2 or perfuming co-ingredients included in mixture 102c do not warrant a more detailed description here, the skilled person being able to select them on the basis of its general knowledge and according to intended use or application and the desired organoleptic effect. In general terms, the flavoring co-ingredients and/or the perfuming co-ingredients belong to chemical classes as varied as alcohols, aldehydes, ketones, esters, ethers, acetates, nitriles, terpenoids, nitrogenous or sulphurous heterocyclic compounds and essential oils, and/or other chemical classes. The flavoring co-ingredients and/or the perfuming co-ingredients may be of natural or synthetic origin. For example, natural flavoring co-ingredients and/or natural perfuming co-ingredients may include essential oils, extracts, and/or products that are derived from a spice, a fruit, a fruit juice, a vegetable, a vegetable juice, an herb, a bud, a root, a leaf, an animal, and/or other natural goods. For example, synthetic flavoring co-ingredients and/or synthetic perfuming co-ingredients may include chemical compositions that are exactly the same as natural flavoring or perfuming co-ingredients, though are produced by chemical synthesis.

Many of these co-ingredients are in any case listed in reference texts such as the book by S. Arctander, Perfume and Flavor Chemicals, 1969, Montclair, N.J., USA, or its more recent versions, or in other works of a similar nature, as well as in the abundant patent literature in the field of flavor and perfumery. It is also understood that said flavoring or perfuming co-ingredients may also be compounds known to release, in a controlled manner, various types of flavoring or perfuming compounds.

In some implementations, flavoring agent 152 may comprise 0.1 to 0.5% by weight of the solution and the other components (e.g., flavor carrier 150, the lubricating agent 170, and/or other components) may accounts for the remaining percentage by weight. In some implementations, a particular amount of the flavoring agent 152 per volume of water and preservatives included in the mixture 102b-c of FIG. 1-2 may resist degradation. For example, degradation can be resisted by making the wipe and/or solution resistant to microbial growth and/or pathogen growth. The amount of flavoring agent 152 added per volume of water (or volume of another non-harmful solution) should be sufficient to provide the pleasant taste. The preservatives may include potassium sorbate, sodium benzoate, decyl glycoside, honeysuckle extract, and/or other preservatives to prevent degradation of the mixture, the solution, and/or disposable cloth 190.

FIG. 4 illustrates a towelette unit, in accordance with one or more implementations. Towelette unit 100 (the same as or similar to FIG. 1-3) may comprise of disposable cloth 190 (the same as or similar to that of FIG. 1-3) that retains a mixture 102d. Mixture 102d may comprise of a combination of flavor carrier 150, flavoring agent 152, perfumery carrier 160, perfume agent 162, lubricating agent 170 and the various implementations described thereof in FIG. 1-3, similar to mixture 102c of FIG. 3. Mixture 102d may further comprise a flavor adjuvant 154 and a perfumery adjuvant 156, and/or other components.

As used herein “flavor adjuvant 154” may refer to an ingredient capable of imparting one or more additional benefits such as a color (e.g., caramel), chemical stability, and so on while simultaneously not imparting or enhancing the taste of the mixture. A detailed description of the nature and type of adjuvant commonly used in flavoring compositions cannot be exhaustive. Nevertheless, flavor adjuvants 154 are well known to a person skilled in the art who will be able to select them on the basis of its general knowledge and according to intended use or application. By way of non-limiting example, flavor adjuvants 154 may include viscosity agents (e.g., emulsifier, thickeners, gelling and/or rheology modifiers, e.g. pectin or agar gum), stabilizing agents (e.g. antioxidant, heat/light and or buffers agents e.g. citric acid), coloring agents (e.g. natural or synthetic or natural extract imparting color), preservatives (e.g. antibacterial or antimicrobial or antifungal agents, e.g. benzoic acid), vitamins and mixtures thereof, and/or other flavor adjuvants. The coloring agents may include dyes, pigments, and/or other coloring agents to provide individual colors.

As used herein “perfumery adjuvant 156” may refer to an ingredient capable of imparting additional added benefit such as a color, a particular light resistance, chemical stability, etc. while simultaneously not imparting or enhancing the odor of the mixture. A detailed description of the nature and type of adjuvant commonly used in perfuming composition cannot be exhaustive, but it has to be mentioned that perfumery adjuvants 156 are well known to a person skilled in the art. For example, the perfumery adjuvants 156 may include viscosity agents (e.g. surfactants, thickeners, gelling and/or rheology modifiers), stabilizing agents (e.g. preservatives, antioxidant, heat/light and or buffers or chelating agents, such as BHT), coloring agents (e.g. dyes and/or pigments), preservatives (e.g. antibacterial or antimicrobial or antifungal or anti irritant agents), abrasives, skin cooling agents, fixatives, insect repellants, ointments, vitamins and mixtures thereof, and/or other perfumery adjuvants.

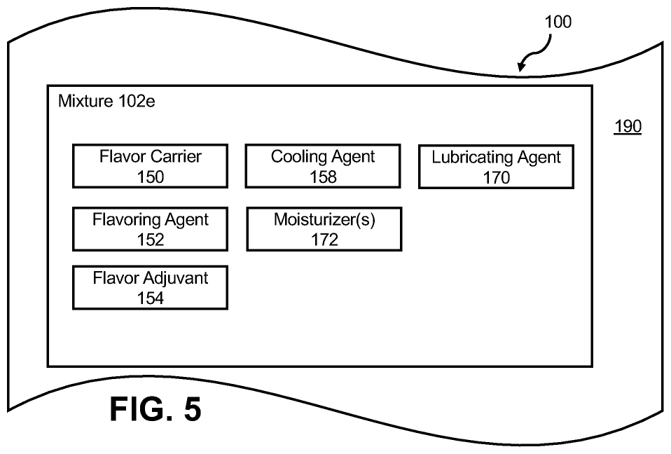

FIG. 5 illustrates a towelette unit, in accordance with one or more implementations. Towelette unit 100 (the same as or similar to FIG. 1) may comprise of disposable cloth 190 (the same as or similar to that of FIG. 1-4) that retains a mixture 102e. Mixture 102e may comprise of a combination of flavor carrier 150, flavoring agent 152, and lubricating agent 170 and the various implementations described thereof in FIG. 2, similar to mixture 102b of FIG. 2, and flavor adjuvant 154 and the various implementations described thereof in FIG. 4. Mixture 102e may further comprise a cooling agent 158, moisturizer(s) 172, and/or other components.

As used herein “cooling agent 158” may refer to compounds that impart a cooling sensation/feeling to the body surfaces. By way of non-limiting example, cooling agents 158 may include WS-23 (2-Isopropyl-N,2,3-trimethylbutyramide), FEMA 3804; WS-3 (N-Ethyl-p-menthane-3-carboxamide), FEMA 3455; WS-5 [Ethyl 3-(p-menthane-3-carboxamido)acetate], FEMA 4309; WS-12 (IR,2S,5R)-N-(4-Methoxyphenyl)-p-menthanecarboxamide, the menthane carboxylic acid esters and amides WS-3, WS-4, WS-5, WS-12, WS-14, WS-30 and mixtures thereof, and/or other cooling agents.

The moisturizer(s) 172 may moisturize and/or soothe the body surface simultaneous to lubrication with the lubricating agent 170. Moisturizer(s) 172 may include glycerin, cocoa butter, aloe vera, and/or other moisturizers. In some implementations, where moisturizing is desired, the amount of moisturizer included in the solution may be selected so as to provide a desired moisturizing capability.

In some implementations, the mixture and thus the towelette unit 100 may include the glycerin in addition to water. The glycerin may be used for its cleaning effect and non-toxic nature if consumed. Glycerin may also provide a moisturizing effect. However, glycerin is believed to have a disagreeable taste or a “soapy” taste to some. Therefore, more of the flavoring agent 152 may be added than that required for water alone to mask the taste of the glycerin, should the glycerin be included.

In some implementations, according to any one of the above implementations, the mixtures 102a-e described may include additional additives. The additives may include vitamin E, chamomile, aloe vera, sugarnate, polysorbate 80, annatto Extract, natural oils (e.g., orange oil), green tea extract, aloe vera juice/extract, and/or other additives for their respective properties they provide. For example, the orange oil may provide a citrus odor and an antibacterial function. For example, the sugarnate, and/or the polysorbate 80 may be used as a surfactant.

According to any one of the above implementations, the mixtures 102a-e of FIG. 1-5 described herein may further comprise ingredients imparting a warming, a tingling, a salivating, or an alcohol enhancement effect such as capsicum extract, spice extract (e.g., ginger, maniguette, all types of peppers including Sichuan, piperine, capsaicine, jambu extract, spilanthol, and/or other ingredients.

In some implementations, the stevia, in particular, may be included in the mixtures 102a-e to provide antimicrobial and anti-pathogen properties to facilitate preservation of the mixtures 102a-e (or In some implementations, the solution) and thus the towelette unit 100 prior to the single-use event. It is noted that sugar or other like sweeteners may leave a sticky residue behind. But the use of stevia (or other sweeteners disclosed herein) can provide the sweet taste without leaving behind a sticky residue on the body surface. The stevia may provide a soothing and/or moisturizing effect to the body surface that counters the effects of a wipe irritating or damaging body surface and may prolong shelf-life by exhibiting antimicrobial and/or anti-pathogen functions.

In some implementations, the xylitol, in particular, may be included in the mixtures 102a-e to provide antimicrobial, anti-bacterial, and/or anti-pathogen properties to facilitate preservation of the mixtures 102a-e (or In some implementations, the solution) and thus the towelette unit 100 prior to the single-use event. The use of the xylitol (or other sweeteners disclosed herein) may provide the sweet taste without leaving behind a sticky residue on the body surface.

The components of mixtures 102a-e and subsequently the solution may be formulated to exist together such that none of the included components will react with any other component to cause a reaction between the components or to cause the degradation of any of the components. This is important because the degradation of the flavoring agent 152 could change the flavor from a pleasant and enjoyable taste to a noxious or unpleasant one. Combining all the components in individual towelette units 100 may allow lubricating, cleaning, and imparting flavors and/or odors to the body surface in a lone event. The simplicity and convenience of the lone event may increase likelihood that users will utilize the towelette units 100.

Flavor carrier 150, the lubricating agent 170, the flavoring agent 152, and/or other components of the mixtures 102a-e as the (liquid) solution may be transmitted from disposable cloth 190 to the body surface by several techniques. Some of the techniques may include the polarity of disposable cloth 190 and the lubricating agent 170, a response to heat, mechanically forcing the lubricating agent 170 from disposable cloth 190, and other techniques.

Transferring the solution from disposable cloth 190 to the body surface by merely wiping the body surface with disposable cloth 190 may provide convenience. The towelette unit 100 may be combined and thus produced without charge. The lubricant (i.e., the lubricating agent 170) will have a charge, so that the lubricant attracts water and keeps it in place on the body surface that the towelette unit 100 wiped. Therefore, the lubricant would easily leave disposable cloth 190 because there is not an attractive force holding it to disposable cloth 190.

In some implementations, the solution (or solutions of individual components) may be added to disposable cloth 190 by the user. The solution(s) may be stored in a solution container such as a vial, a capsule, a spray canister, a bottle, or other solution container. The solution may be added to disposable cloth 190 by, for example, spraying or pouring the solution(s) from the solution container. In some implementations, the solution may be emulsified and dispensed as a cream or lotion by a pump dispenser. The emulsified solution may also be dispensed from a squeezable tube having a small hole (or multiple holes in the cap), or may be provided in a “makeup” type compact for dipping disposable cloth 190 into.

In some implementations, disposable cloth 190 may include multiple layers of cloth. The solution may be retained in one of the layers. The disposable cloth 190 may not be limited to any particular number of layers nor is disposable cloth 190 limited to having separate layers. Also incorporated by reference in their entirety are U.S. Patent Publication No. 2002/0127937 to Lange et al., U.S. Patent Publication No. 2006/0151518 to Sarbo et al., U.S. Pat. No. 6,361,784 to Brennan et al. and U.S. Patent Publication No. 2012/0090113 to Manifold.

To prepare disposable cloth 190 with the solution, one may submerge a roll of multiple ones of disposable cloths 190 or individual disposable cloths 190 within the solution and allow it to soak for a sufficient time period to allow the solution to be imparted onto, impregnated within, or retained by disposable cloths 190. Alternatively, the solution may be sprayed, pumped, or poured onto disposable cloth 190. Individual disposable cloths 190 of the roll may extend to a pre-formed length and width (e.g., 5 inches, 8 inches, etc.). This is currently well-known by those of ordinary skill in the art.

In some implementations, the towelette units 100 may be packaged in a ready to use state. Being in a ready to use state the wipes will be packaged such that they can be used immediately upon removal from the package. This is advantageous for increasing the likelihood of being used as well. Being packaged in a ready to use state also requires packaging that prevents desiccation or drying out of the towelette units 100.

In some embodiments the mixture and thus the solution (upon the dry mixture getting wet) may be retained by the entirety of disposable cloth 190. In some implementations, the individual components such as the lubricating agent 170, flavor carrier 150, the flavoring agent 152, and/or other components may be liquids and may be segregated to portions of disposable cloth 190. In some implementations, individual components such as the lubricating agent 170, flavor carrier 150, the flavoring agent 152, and/or other components may be dry and coupled with disposable cloth 190 in segregated portions of disposable cloth 190.

For example, some users may want to know that the body surface is cleansed prior to application of flavor carrier 150 and the flavoring agent 152, and that the lubrication is applied after the application of flavor carrier 150 and the flavoring agent 152. In this way a single towelette unit may be used for the individual applications of the individual components. For example, one partner could apply the cleaning agent, then flavor carrier 150 and the flavoring agent 152, then the lubricating agent 170 as each of the components are necessary.

By way of non-limiting illustration, the portion half a single disposable cloth 190 may be colored blue (e.g., with the coloring agent described herein) to indicate use for cleansing and flavoring (and thus include the cleaning agent, flavor carrier 150, and the flavoring agent 152) and the other half of the same disposable cloth 190 may be colored pink to indicate use lubrication (and thus include the lubricating agent 170). Though the disposable cloth 190 in this example utilize two agents for performing separate steps, the towelette units 100 may be in a ready to use state similar to that described above.

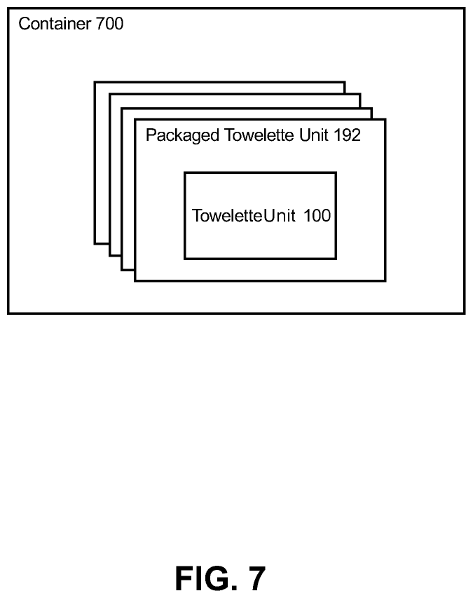

FIG. 7 illustrates a towelette container configured to store individually packaged towelette units, in accordance with one or more implementations. FIG. 7 may illustrate container 700 that is configured to store individually packaged towelette units 192. Individual ones of the packaged towelette units 192 may include an individual towelette unit 100 (the same as FIG. 1-5) and packaging that encloses the individual towelette units 100. The packaging may include one or more of paper packaging material, plastic packaging material, foil sealed packaging material, and/or other packaging materials. As such, the towelette units 100 may be separate and distinct from other ones of the towelette units 100 and facilitate immediate access and use thereof. Towelette units 100 may include a flavor carrier, a lubricating agent, a disposable cloth, and/or other components (as in FIG. 1-5). As previously described, at least the flavoring carrier and the lubricating agent and/or other components may be combined to create a mixture (See, mixture 102a-e of FIG. 1-5). The disposable cloth may be configured to retain the mixture for a subsequent single-use event (as described in FIG. 1-5).

In some implementations, the mixture may be a solution (as described above) and the disposable cloth may be saturated with the solution. The packaging (e.g., the foil sealed packaging material) may facilitate preservation of the solution until a single-use event.

In some implementations, the container 700 may be configured to store and/or dispense the saturated towelette units 100, with or without individual packaging. In some implementations, the packaged towelette units 192 may be arranged as a roll of individually packaged towelette units. The individual packaged towelette units 192 may be detachable from the roll by a perforation between the individual packaged towelette units 192.

In some implementations, the disposable cloths may be saturated in the solution in the container 700. In some implementations, the towelette units 100 may be arranged as a roll of towelette units. The roll may be stored in container 700. The individual towelette units 100 may be detachable from the roll by a perforation between the individual towelette units to facilitate distribution and use thereof. The container 700 may include an opening to provide access to the towelette units 100 or packaged towelette units 192. The opening may be sealable to shield the towelette units 190 from external elements.

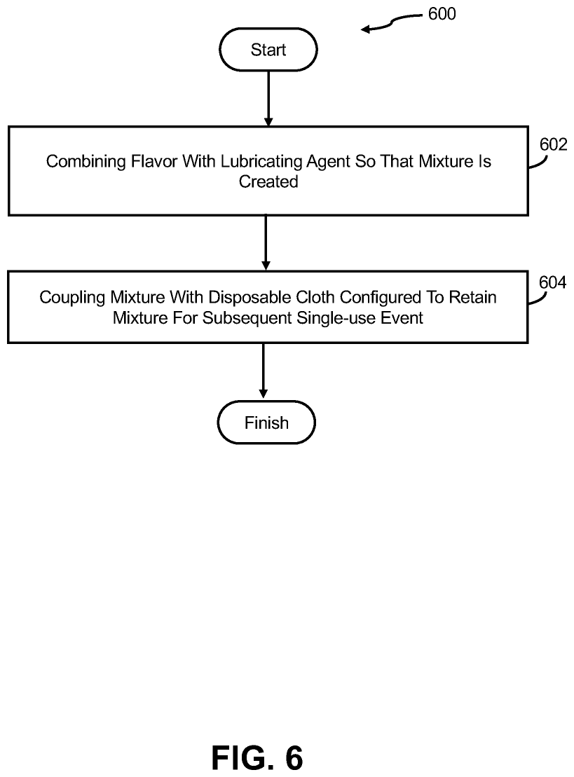

FIG. 6 illustrates a method for providing a towelette unit, in accordance with one or more implementations. The operations of method 600 presented below are intended to be illustrative. In some implementations, method 600 may be accomplished with one or more additional operations not described, and/or without one or more of the operations discussed. Additionally, the order in which the operations of method 600 are illustrated in FIG. 6 and described below is not intended to be limiting.

An operation 602 may include combining a flavor carrier that facilitates transmitting a flavoring agent to a body surface upon contact with a lubricating agent that reduces friction between body tissues so that a mixture is created.

An operation 604 may include coupling the mixture with a disposable cloth, wherein the disposable cloth is configured to retain the mixture for a subsequent single-use event, wherein the single-use event includes wiping the body surface with the disposable cloth.

It is understood that a person skilled in the art is perfectly able to design optimal formulations for the desired effect by admixing the above-mentioned components of a flavoring, perfuming, and/or lubricating mixture, simply by applying the standard knowledge of the art as well as by trial-and-error methodologies.

For the sake of clarity, it is also understood that any mixture resulting directly from a chemical synthesis, e.g., a reaction medium without an adequate purification, in which the compounds of the invention would be involved as a starting, intermediate or end-product could not be considered as a flavoring, perfuming, or lubricating mixture according to the invention as far as said mixture does not provide the inventive compound in a suitable form for perfumery or for flavor. Thus, unpurified reaction mixtures are generally excluded from the present invention unless otherwise specified.

All patents and published patent applications referred to herein are incorporated herein by reference. However, any reference to prior publication is not, and should not be taken as an acknowledgement, admission, or suggestion that the prior publication, or any information derived from it is part of the general common knowledge in the field of endeavor to which this specification relates. The invention has been described with reference to various specific and preferred embodiments and techniques. Nevertheless, it is understood that many variations and modifications may be made while remaining within the spirit and scope of the invention.

1. (canceled)

2. (canceled)

3. The towelette unit of claim 5, wherein the mixture further comprises a perfume agent that provides a scent, wherein the perfume agent is natural and/or synthetic.

4. (canceled)

5. A towelette unit, the towelette unit comprising:
a flavor carrier that facilitates transmitting a flavoring agent to a body surface upon contact;
the flavoring agent that provides a sour taste;
a lubricating agent that reduces friction between body tissues and facilitates water retention on the body tissues, wherein at least the flavoring carrier, the flavoring agent, and the lubricating agent are combined to create a mixture; and
a disposable cloth individually packaged separate and distinct from other disposable cloths, wherein the individual disposable cloths are packaged in one or more of paper packaging material, plastic packaging material, and/or foil-sealed packaging material that are corrupted and unusable upon opening to remove disposable cloth, wherein the individual disposable cloths are configured to retain the mixture for a subsequent single-use event, wherein the single-use event includes wiping the body surface with the disposable cloth, wherein the disposable cloths are comprised of polyester fabrics, rayon fibers, or both, wherein the mixture is a dry mixture coupled to the disposable cloth, wherein upon application of a liquid to the dry mixture coupled with the disposable cloth, a solution is created and retained by the disposable cloth.

6. The towelette unit of claim 5, wherein the lubricating agent further comprises one or more humectants, wherein upon wiping the body surface with the towelette unit, the one or more humectants are applied to the body surface.

7. The towelette unit of claim 5, wherein the mixture further comprises xylitol to provide antimicrobial and anti-pathogen properties to facilitate preservation of the solution prior to the single-use event.

8. The towelette unit of claim 5, wherein the mixture further comprises one or more sweeteners that provide a sweet taste and/or sweet odor, wherein the one or more sweeteners include xylitol, wherein the flavoring agent includes the one or more sweeteners.

9. The towelette unit of claim 5, where the mixture further comprises one or more moisturizers to simultaneously moisturize the body surface while lubricating with the lubricating agent.

10. (canceled)

11. (canceled)

12. The method of claim 14, wherein the mixture further comprises a perfume agent that provides a scent, wherein the perfume agent is natural and/or synthetic.

13. (canceled)

14. A method to provide a towelette unit, the method comprising:
combining a flavor carrier, a flavoring agent, and a lubricating agent so that a mixture is created, wherein the flavor carrier facilitates transmitting the flavoring agent to a body surface upon contact, wherein the flavoring agent provides a sour taste, wherein the lubricating agent reduces friction between body tissues and facilitates water retention on the body tissues; and
coupling the mixture with a disposable cloth that is individually packaged separate and distinct from other disposable cloths, wherein the individual disposable cloths are packaged in one or more of paper packaging material, plastic packaging material, and/or foil-sealed packaging material that are corrupted and unusable upon opening to remove disposable cloth, wherein the individual disposable cloths are configured to retain the mixture for a subsequent single-use event, wherein the single-use event includes wiping the body surface with the disposable cloth, wherein the disposable cloths are comprised of polyester fabrics, rayon fibers, or both, wherein the mixture is a dry mixture coupled to the disposable cloth, wherein upon application of a liquid to the dry mixture coupled with the disposable cloth, a solution is created and retained by the disposable cloth.

15. (canceled)

16. (canceled)

17. (canceled)

18. (canceled)

19. (canceled)

20. (canceled)

21. The method of claim 14, wherein the lubricating agent further comprises one or more humectants, wherein upon wiping the body surface with the disposable cloth that retains the solution, the one or more humectants are applied to the body surface.

22. The method of claim 14, further comprising combining xylitol with the flavor carrier and the lubricating agent to provide antimicrobial and anti-pathogen properties to facilitate preservation of the solution prior to the single-use event.

23. The method of claim 14, further comprising combining one or more sweeteners with the flavor carrier and the lubricating agent that provide a sweet taste and/or sweet odor, wherein the one or more sweeteners include xylitol, wherein the flavoring agent includes the one or more sweeteners.

24. The method of claim 14, further comprising combining one or more moisturizers with the flavor carrier and the lubricating agent to simultaneously moisturize the body surface while lubricating with the lubricating agent.

25. (canceled)

26. (canceled)
